(19) United States
(12) Patent Application Publication
(10) Pub. No.: US 2022/0395331 A1
(43) Pub. Date: Dec. 15, 2022

(54) DIAGNOSTIC METHOD FOR MIGRAINE HEADACHES

(71) Applicant: Kevin Raynard Smith, Houston, TX (US)

(72) Inventor: Kevin Raynard Smith, Houston, TX (US)

(21) Appl. No.: 17/892,763

(22) Filed: Aug. 22, 2022

Related U.S. Application Data
(63) Continuation of application No. 16/942,227, filed on Jul. 29, 2020, now abandoned.
Application No. 16/942,227 is a continuation of application No. 16/927,894, filed on Jul. 13, 2020, now abandoned.
Application No. 16/927,894 is a continuation of application No. 15/628,623, filed on Jun. 20, 2017, now abandoned.
(60) Provisional application No. 62/352,475, filed on Jun. 20, 2016.
(60) Provisional application No. 62/352,473, filed on Jun. 20, 2016.

(51) Int. Cl. A61B 34/10; A61B 6/00; G06T 7/00; G06T 7/11; A61B 5/00; A61B 5/08

(52) U.S. Cl. CPC A61B 34/10; A61B 6/5217; G06T 7/0012; G06T 7/11; A61B 5/0037; A61B 5/0042; A61B 5/08; A61B 5/4824; A61B 5/4836; A61B 5/4887; A61B 2034/101; A61B 2090/3762

(57) ABSTRACT

A diagnostic method for determining if a patient is viable candidate for a surgical procedure to permanently eliminate migraine headache pain. The method includes determining if a patient's pain is a migraine pain condition or pain from another medical condition and determining an anatomical location of the determined migraine pain condition. Once the anatomical location is determined, the method correlates the determined anatomical location of the migraine pain condition to a root cause nasal/sinus location and determines a root cause nasal/sinus condition that is causing the patient's migraine pain. Once these diagnostic procedures are complete, the patient may be scheduled for surgery to eliminate the root cause nasal/sinus condition, and thus, permanently eliminate the migraine headache pain.

### CROSS-REFERENCE TO RELATED APPLICATIONS

This application claims the benefit of U.S. application Ser. No. 16/927,894, filed Jul. 13, 2020. This application is incorporated herein by reference in its entirety to the extent consistent with the present application

### FIELD

Embodiments of the disclosure relate to a diagnostic method for determining if a patient is a candidate for a particular type of migraine treatment that permanently eliminates migraine headache pain.

### BACKGROUND

A need exists, a need in the medical field for a diagnostic process or method for determining if a patient suffering from chronic headaches or migraine is a viable candidate for successful nasal/sinus treatment to permanently eliminate the migraine and chronic headaches.

Migraines are a common neurologic disease that affects over a billion people globally. In the United States, migraines are estimated to impact over 12% of the population and this impact is severe. Migraines are debilitating, underdiagnosed, and subject the patient to years of ongoing and harmful treatments, rarely permanently eliminating the migraines. Migraines are one of the most burdensome neurologic diseases, as headache disorders (including migraines) are the second leading cause of years lost to disability worldwide, second only to lower back pain. Further, more than half of migraine patients experience severe impairment of daily activities or a need for significant bed rest during a migraine attack. Family and work activities are severely disrupted and substantial economic burdens are imposed on society through increased health care costs and lost productive working days. As an example, patients with migraines lose an average of 2-4 work days every month also costing business billions annually.

Migraine diagnosis typically begins with a neurologist's review of the patient's family history and a review of the patient's symptoms along with a physical and neurological examination. Medical tests to rule out other causes of a patient's pain, typically include MRI and CT scans to produce images of the brain and blood vessels to look for tumors, strokes, bleeding in the brain, infections, and other brain and neurological conditions that are known to cause similar pain characteristics as migraines. Only 5% of these brain scans will show abnormalities in the brain as a cause for head pain. The remaining 95% of patients with a negative brain scan are diagnosed with migraines and/or chronic headaches. When a migraine is determined, then current migraine treatments are focused on stopping symptoms and reducing future attacks through pain relieving medications (abortive treatment to stop symptoms during a migraine attack) and preventative medications intended to reduce the severity or frequency of migraines. Exemplary medications used to treat the symptoms of migraines include common over the counter pain relievers, triptans that block pain pathways to the brain, dihydroergotamines that narrow blood vessels around the brain to affect blood flow patterns, lasmiditan works on serotonin receptors on nerves that are generally inside the brain itself that are responsible for transmitting the pain of headache, ubrogepant is an oral calcitonin gene-related peptide receptor antagonist, opioid medications and especially those that contain codeine may help migraines (despite their highly addictive nature), and anti-nausea medications have been shown to reduce migraine intensity (pain levels) while also reducing the frequency of migraines.

Other treatments for migraines include but are not limited to, Botox injections, biofeedback, hypnosis, massage, chiropractic treatment, nerve block, acupuncture, lumbar puncture, nerve decompression, hysterectomy and placement of a spinal stimulator.

Each of the above noted treatments have been shown to sometimes reduce the severity of migraines, and in some cases, the treatments have also been shown to sometimes reduce the frequency of migraines. However, there is currently no known migraine treatment that has been shown to be capable of permanently eliminating migraines for patients. In view of the vast number of people worldwide that suffer from migraines, there is a clear need for a medical treatment that is capable of permanently eliminating migraines for patients. Further, there is a need for a diagnostic method for identifying the root cause of migraine headaches and determining if a patient is a candidate for a permanent migraine eliminating medical treatment.

Embodiments of the present disclosure provide a novel medical treatment capable of identifying and removing the root cause of migraines, permanently eliminating/curing migraines along with a diagnostic method for determining if a patient is a candidate for the novel migraine treatment procedure.

### BRIEF DESCRIPTION OF THE DRAWINGS

The detailed description will be better understood in conjunction with the following drawings:

FIG. 1 illustrates a front view of a congested sinus with a bone spur according to one or more embodiments.

FIG. 2 is a front view of a head of a patient showing sinus cavities according to one or more embodiments.

FIG. 3 is a side view of nasal cavities of a patient according to one or more embodiments.

FIG. 4 illustrates a computer tomography scan of congested nasal and sinus cavities according to one or more embodiments.

FIG. 5 illustrates a computer tomography scan of anesthetized decongested nasal cavities according to one or more embodiments.

FIG. 6 illustrates a kit with equipment used to implement migraine reduction according to one or more embodiments.

FIG. 7 illustrates a data storage according to one or more embodiments.

FIG. 8 illustrates an exemplary diagnostic method of the present disclosure.

### DETAILED DESCRIPTION

Embodiments of the present disclosure relate to a novel medical treatment capable of permanently eliminating/curing migraines along with a diagnostic method for determining the root cause of the migraines and if a patient is a viable candidate for the novel migraine treatment procedure.

Overall, embodiments of this disclosure provide a diagnostic method for determining the root cause of migraine and if a patient is a viable candidate for a medical procedure to permanently eliminate migraine headache pain. The diagnostic method begins with determining if a patient's pain is a migraine pain condition or pain from another medical condition, and then determining the nasal/sinus anatomical locations of the determined migraine pain condition. Once the anatomical location is determined, which is typically a region in the head, the method correlates the determined anatomical location of the migraine pain condition to a root cause sinus location (a specific sinus location) and determines a root cause nasal/sinus condition (a turbinate contacting adjacent structures, a deviated septum, a bone spur, etc.) that is causing the patient's migraine pain. Once these diagnostic procedures are complete, the patient may be scheduled for surgery to eliminate the root cause nasal/sinus condition, and thus, permanently eliminate the migraine headache pain.

The first step of treatment of a migraine is diagnosing the headache pain as a migraine, as there are numerous types of headaches, including tension, cluster, allergy, hormone, caffeine, exertion, and hypertension, for example. Further, headaches can generally be classified into three types: 1) primary headaches; 2) secondary headaches; and 3) cranial neuralgias, facial pain, and other headaches. Migraine, tension, and cluster headaches are common types of primary headaches. Secondary headaches are those that are due to an underlying structural or infectious problem in the head or neck that may include dental pain from infected teeth, an infected sinus, brain bleeding, and/or brain infections.

Diagnosing a headache as a migraine may include a thorough review of a patient's (and often times family's) medical history with a focus on prior headaches location, intensity, frequency, and duration. In addition to medical history, a neurologist will perform a complete physical and neurological exam looking for signs and symptoms of an illness or other condition that causes migraine headaches. The physical exam may focus on conditions such as fever, abnormalities in breathing, pulse, or blood pressure, infections, nausea, mental acuity, recent behavior changes, seizures, fainting, fatigue, weakness, numbness, tingling, speech difficulty, balance problems, dizziness, and/or vision changes. These neurological tests and conditions focus on identifying and/or ruling out diseases of the brain or nerves that are known to be causes of more severe headaches. Since most headaches are benign and not a migraine, many of the neurological tests look for a physical or structural abnormality in the brain that may cause a headache, such as tumors, abscesses, hemorrhage, bacterial or viral infections, intercranial pressure, fluid buildup, swelling of the brain, head trauma, blood vessel abnormalities, and aneurysms. When these conditions are found, then the likelihood that the patient has a migraine is substantially reduced, as these conditions have been shown to cause headaches that can be mistaken for migraines.

Once the headache is determined to be a primary headache (migraine, cluster, or tension headache), then the diagnostic process of the present disclosure continues to determine the location of the pain condition resulting from the headache. Location of pain may be determined, for example, from patient survey information, from physical exam observations, review of scans or images, etc.

Once the location of the headache pain is determined, then the location of the headache pain is correlated to a source or root cause nasal/sinus location. For example, if a patient has a primary headache and pain in their teeth, then the headache pain may be correlated to the maxillary sinus as being the root cause. Similarly, temple pain may be correlated to the inferior turbinates and/or the ostium or opening of the maxillary sinus. Pain around, between, or behind the eyes may be correlated to the middle turbinate or ethmoid sinus. Perinasal pain may be correlated to the middle turbinate or ethmoid sinus. Pain on the top of the head may be correlated to the sphenoid sinus. Ear pain may be correlated to inferior turbinate enlargement impairing the eustachian tube or a septal deviation or spur touching the middle turbinate. Pain with dizziness may be correlated to eustachian tube dysfunction related to large inferior turbinates and/or a deviated septum. Tinnitus or ringing in the ears and/or intermittent hearing loss may also be correlated to eustachian tube dysfunction related to large inferior turbinates and/or a deviated septum.

As background, turbinates, which are also called nasal concha or conchae (plural), are shell-shaped networks of bones, vessels, and tissue within the nasal passageways. These structures are located on the lateral aspect of the nasal cavity and are responsible for warming, humidifying, and filtering the air we breathe. Normally there are three paired turbinates including the superior (upper), middle, and inferior (lower) turbinates. However, occasionally you can have a fourth turbinate (called the supreme turbinate) which is situated higher than the superior turbinate. In between each turbinate is space (known as meatus), each with a name that coincides with the name of the turbinate that is directly above the space. These spaces help form our nasal passageways which direct air flow through our nose.

As background, on the sinus locations, the middle meatus is the nasal passageway that lies between the inferior and middle turbinate. This space functions to assist with drainage of three of the paranasal sinuses; the maxillary, frontal, and front (anterior) ethmoid sinuses. It also allows airflow through the paranasal sinuses which creates the tones of voices. The Superior meatus is the nasal space that lies between the middle and the superior turbinates. This is normally the top-most nasal passageway (occasionally there is also a supreme meatus that is between the superior and supreme turbinates). Functions of this passageway include drainage of two of the paranasal sinuses: the sphenoid and back (posterior) ethmoid sinuses; allowing airflow through the passageway (which interacts with sinus cavities) helps to modify our vocal features; and mucous membranes of the superior turbinate (along with the upper part of the nasal septum, which divides the left and right nostrils) are lined with nerve endings from the olfactory nerve which are used to interpret smell. The upper and middle conchae are part of the ethmoid bone, but the lower conchae is an independent structure. The inferior meatus is the space between the floor of the nasal cavity and the inferior turbinate. This is the largest of the air spaces. This passageway serves multiple purposes, including; nasolacrimal duct (tear duct) empties any drainage from the eyes, starting at the external eye and emptying into the inferior meatus; and the head of the nasal wall, inferior meatus, and osseous piriform aperture make up the nasal valve, which is the narrowest area in the nasal cavity and is often the site of obstruction (collapse) due to a deviated septum or other nasal abnormalities.

Once the location of the headache pain is correlated to a source or root cause nasal/sinus location in the diagnostic method of the present disclosure, then the root cause nasal/sinus location is analyzed to determine a nasal/sinus condition causing the headache pain. Analyzing the root cause sinus location to identify or determine a root cause condition generally entails the use of imaging techniques, such as radiographic imaging of which CT scan of the head and general sinus areas is the current standard. These CT scans are analyzed to identify particular physical conditions that the novel method of the present disclosure uses to correlate headache pain and specifically migraine pain to a root cause condition, i.e., specific physical conditions in the nasal/sinus areas can be correlated via the novel method of the present disclosure to a root cause of specific migraine headache pains.

Analyzing the nasal/sinus area on CT scan is an involved and intricate process. The first step to sinus CT scan review as part of the present novel methodology is knowing the patient's medical history, as described above. Generally, the most efficient way to obtain this information is using a comprehensive assessment of the patient's symptoms to identify the sinus area of concern prior to reviewing the actual CT scan images of the identified sinus area. Preferably, the CT scan radiographic images should be a high-resolution CT scan of the sinuses without contrast with slices of less than one millimeter.

The midline structures of the nose may be evaluated in a manner where the nasal septum is examined for deviations or bone spurs. The nasal turbinates may be examined, starting inferiorly and then progressing superiorly, for hypertrophy and any contact with the septum or the confines of the nasal cavity (floor or lateral nasal wall). In addition to analyzing for turbinate contact, the method of the present disclosure analyzes the curvature of the inferior turbinates, as curvature on the inferior margin as opposed to a generally straight downward hanging configuration in the nasal cavity, generally termed the “elf shoe” deformity, are noted as part of the diagnostic method of the disclosure. Middle and superior turbinates may also be analyzed for hypertrophy and/or contact with the septum or other adjacent anatomical structures in the nasal cavity and are noted as part of the diagnostic method of the disclosure. The diagnostic method may further include determining the presence of a concha bullosa, an air cell within the middle or superior turbinates. The diagnostic method analyzes the paranasal sinuses, including the frontal sinuses to determine if mucosal thickening or opacification is present, if there is narrowing or obstruction of the ostium or drainage tract, and if scarring from prior surgical treatment or trauma is present. The diagnostic method analyzes the maxillary sinuses for mucosal thickening and/or opacification and looks for a narrowed or obstructed ostiomeatal complex or impaired drainage caused by an enlarged middle turbinate or polyp. For the sphenoid sinuses, the method analyzes the amount of mucosal thickening as well as obstruction or narrowing of the ostia. The spheno-ethmoid recess is evaluated by the method of the present disclosure for narrowing or obstruction by the hypertrophic superior turbinates, which is best seen on the axial view of the sinus CT scan.

The diagnostic method of the present disclosure, when analyzing the CT scan images of the nasal cavity, is generally configured to determine boundaries of the turbinates and determine if those boundaries touch any portion of the sensitive mucosa comprising the turbinates, as this type of turbinate contact with it's boundaries has been shown by the inventors to be a root cause of migraine headaches. Recognizing the nasal respiratory cycle by which the right and left turbinates alternately swell and shrink is also important as there may be permanent indentions on a decongested turbinate due to contact with a septal deviation during the swollen phase. There may not be any septal turbinate contact at the time of the scan. Swollen, enlarged or hypertrophy turbinates can contact the septum or other boundaries of the nasal cavity causing headaches or migraines. Further, diagnostic method of the present disclosure, when analyzing the CT scan images of the various sinus cavities and nose, is also generally configured to review turbinate shape for abnormalities that have been shown by the inventors to be a root cause of migraine headaches. More particularly, aside from the shape of turbinates causing physical contact with adjacent structures, the shape of turbinates being significantly deformed or altered from the original shape has also been shown by the inventors to be a root cause of migraine headaches. The “elf shoe” turbinate deformity (curvature of the inferior turbinates) has also been shown by the inventors to be a root cause of migraine headaches.

The determination of boundaries for turbinates and the surrounding structures may be accomplished by a software process or program product. For example, a software program may be configured to identify exterior boundaries for turbinates on a CT scan by analyzing the difference in pixel color on the CT scan at the edge of the turbinates so as to map an exterior boundary of a turbinate at the pixel boundary with the greatest disparity. Similarly, the boundary of adjacent nasal tissue (adjacent to the turbinate) may also be identified by the software process of the present embodiment. Once the exterior boundaries of the turbinates and the adjacent tissue have been identified, the software may also analyze the boundaries to identify intersections or areas where the boundaries are in close proximity to each other, thus identifying areas where turbinates are likely to touch adjoining nasal tissue and therefore become a root cause of migraine pain for the patient. A similar pixel disparity analysis that defines boundaries for various tissues may be used to identify a deviated septum, a bone spur, and other sinus conditions noted above that the novel method of the present disclosure has found are root causes of migraine pain.

Once the diagnostic method of the present disclosure identifies the nasal/sinus migraine root cause condition(s), which are generally nasal/sinus abnormalities, turbinate deformity, turbinate contact with adjacent structures, septal deviations/bone spurs, sinus disease and sinus outflow tract and/or ostial narrowing or obstruction, then the method of the present disclosure continues to the treatment phase. The treatment phase generally includes surgically repairing or correcting the identified root cause abnormalities restoring the nose and sinus anatomy to a normal configuration, including separating turbinates from contacting adjacent structures, reducing or trimming enlarged or deformed turbinates to remove the deformity, correcting or removing a deviated septum/bone spur in the nose, or dilating narrowed or obstructed sinus ostia. It should be noted, however, that the nasal/sinus root cause conditions causing severe pain may not be considered a migraine but a chronic headache by some clinicians, however, despite the terminology used or the selected definition of a migraine, the novel method of the present disclosure allows a medical practitioner to identify nasal/sinus conditions and/or abnormalities that when addressed or corrected with minor surgery can completely and permanently eliminate migraine severe headache pain for the patient. To achieve success in eliminating migraines via nasal/sinus surgery, thorough understanding of the correlation between abnormal nasal/sinus anatomy and its effect on migraine and severe headache must be achieved, and proper surgical technique to eliminate migraine must be mastered.

Turning to surgical procedures that are used to correct root cause conditions that result in patient migraines, a septoplasty, for example, is a surgical procedure to straighten the bone and cartilage dividing the space between the two nostrils (septum). A septoplasty is generally performed by making an incision in the anterior mucosa of the septum and elevating the mucosa off the deflected cartilage and/or bone. The crooked septum and/or spurs are removed taking care to keep the nasal support intact. In cases where the caudal septum is destabilized, intranasal splints may be used. Reduction of the inferior turbinate may be achieved with a variety of methods including ablation of the soft tissue when the turbinates are mildly to moderately enlarged and cause mild impairment of breathing. For large inferior turbinates that cause moderate to severe nasal obstruction or ear problems (hearing loss, tinnitus, dizziness or pain), partial turbinate excision may be conducted. Enlarged middle turbinates can make contact with the septum and/or impair the drainage of the frontal and maxillary sinuses. Depending on turbinate size, treatments can range from conservative to aggressive. These turbinates can be gently out fractured laterally away from the septum when contact is present. If mildy enlarged due to mucosal hypertrophy they can be reduced by ablation. When the major component causing the enlargement is bone, they can be reduced by squeezing with a hemostat. Large pedunculated middle turbinates, whether from mucosa, bone or concha bullosa, can be excised. Superior turbinates which make contact with the upper septum are treated by gently pushing them laterally from the septum protecting the branches of the olfactory nerve to preserve the sense of smell.

For abnormalities of the frontal, maxillary or sphenoid sinuses, a balloon sinuplasty may be performed to dilate the natural outflow tracts to allow for proper drainage and aeration. Currently, the Acclarent Balloon Sinuplasty apparatus is considered the best by the inventor. The respective guide catheters are used on the sinuses, the guidewire with a light on the tip is used to enter the sinuses through the natural tract and opening. Placement is confirmed by the illumination of the sinuses. Once in place, the balloon is passed over the wire and inflated to 12 mm of water pressure to dilate the ostia or tract, then deflated. With the catheter in place, the sinuses are irrigated with saline. The catheter is then removed. The ethmoid sinuses are surgically treated when opacified or they have mucosal thickening that correspond to complaints of pain between or behind the eyes. These honeycomb cells are removed with small sinus biting instruments to create one common cavity. The anterior ethmoid can be treated alone or the entire anterior and posterior ethmoid cells can be removed depending on the involvement on the sinus CT scan.

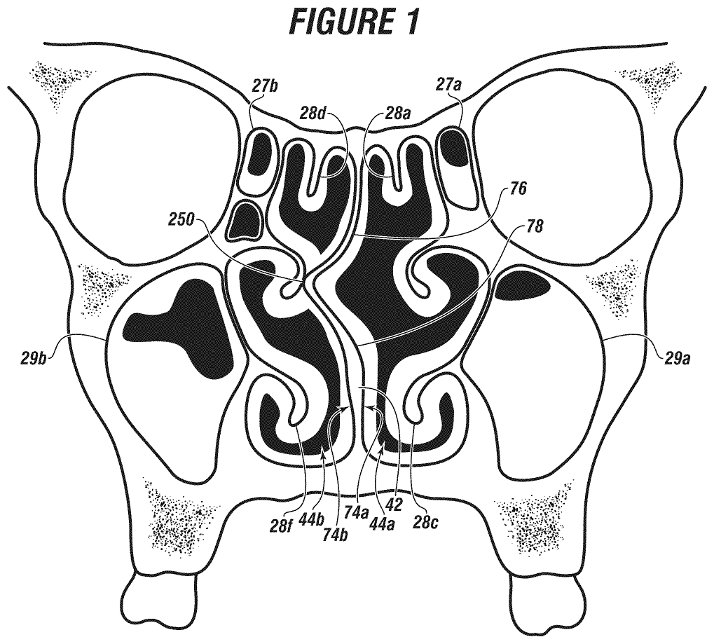

FIG. 1 illustrates a coronal (front) view of a congested sinus with a bone spur and other abnormalities or potential migraine causing conditions according to one or more embodiments of the disclosure. The coronal (front) view may be of a generally vertical slice of a CT scan image, wherein vertical refers to a plane through the patient's ears that is generally perpendicular to the floor when a person is standing upright. The patient can have at least one turbinate 28a-28f, which are also called nasal concha or conchae, and are generally shell-shaped networks of bones, vessels, and tissue within the nasal passageways. These turbinate structures function to warm, humidify, and filter the coming into the nose passages (the air patients breathe). FIG. 1 also illustrates a septal bone spur 250 that substantially deforms the path of the septum 42 toward the left side of the Figure and touches the middle turbinate, which under normal conditions (without a bone spur 250 or other trauma to the nose area) the septum 42 would be generally be a straight line going up and down in the Figure. Mucosal flaps 74a and 74b are shown at the base of the septum, an upper septal bone 76, a segment of septal cartilage 78, a pair of ethmoid sinuses 27a and 27b, at least one maxillary sinus 29a and 29b, a nasal septum 42, nasal cavities 44a and 44b, or combinations thereof.

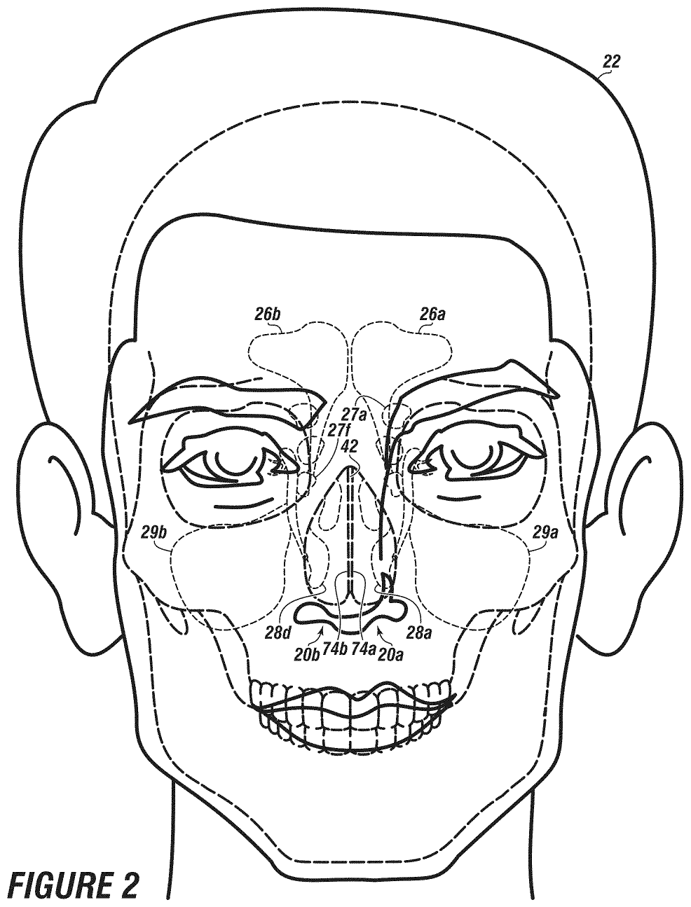

FIG. 2 is a front view of a head of a patient showing the nose and sinus cavities. The patient 22 can have a first mucosal flap 74a and a second mucosal flap 74b of a nasal septum 42, at least one frontal sinus cavity 26a and 26b, a pair of ethmoid sinuses 27a and 27f, at least one superior turbinate 28a and 28d, and at least one maxillary sinus 29a and 29b. The surgical treatment of the headaches, namely migraine headaches, can be accessed through at least one nostril 20a and 20b.

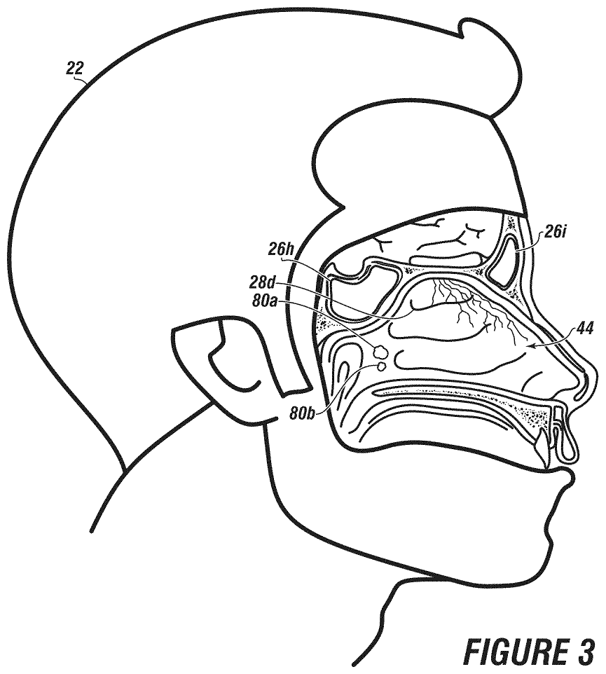

FIG. 3 is a side view of nasal cavities of a patient 22 according to one or more embodiments of the disclosure. The patient 22 can have at least one sinus cavity, such as the sphenoid sinus cavity 26h, a frontal sinus cavity 26i and at least one superior turbinate 28d in the nasal cavity 44. In this embodiment, the nasal cavity 44 is shown with unwanted matter 80a and 80b.

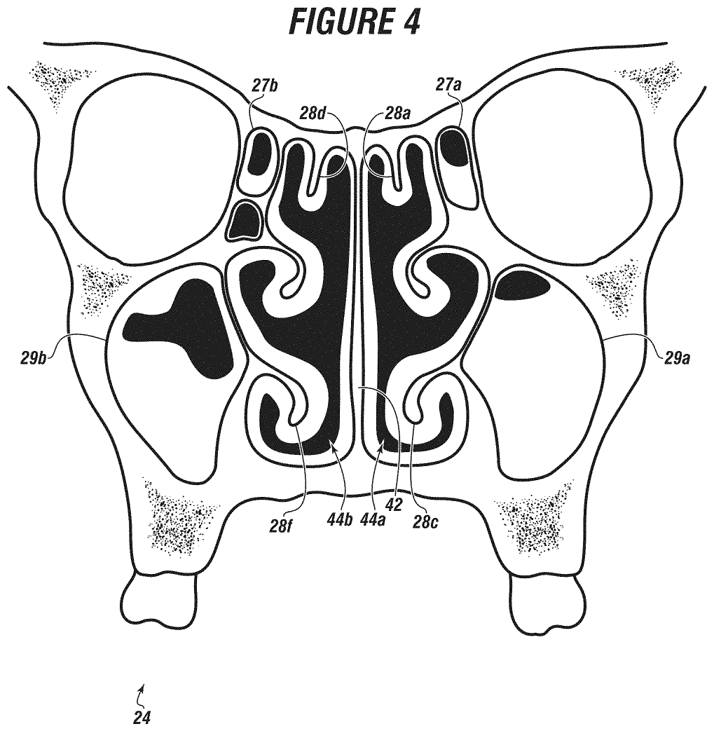

FIG. 4 illustrates a computer tomography scan 24 of congested nasal and sinus cavities according to one or more embodiments. The nasal cavity shown in FIG. 4 generally illustrates turbinates 28a-28f, in their normal position, i.e., in a position that is not a root cause of migraine headaches. This is generally due to the turbinates hanging normally in a position where they are not touching any of the adjoining or nearby structures and not being enlarged or otherwise not of a normal size, shape, or configuration. The nasal septum 42 is relatively straight without deviation or bone spur touching the turbinates, i.e., in a position that is not a root cause of migraine headaches.

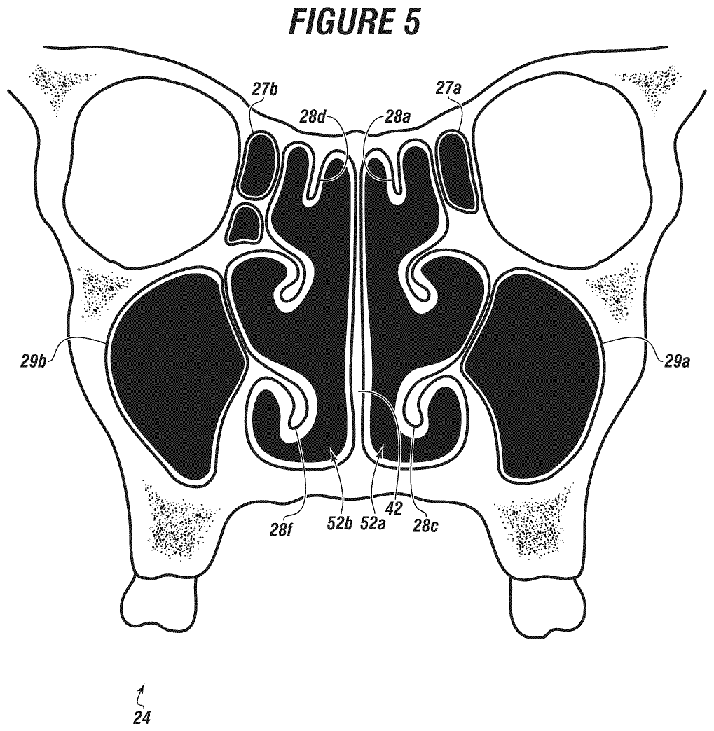

FIG. 5 illustrates a computer tomography scan of anesthetized decongested nasal cavities according to one or more embodiments. The computer tomography scan 24 of anesthetized decongested nasal cavities 52a and 52b are shown. In embodiments, the computer tomography scan can show the at least one turbinate 28a-28f, the at least one maxillary sinus 29a and 29b, the pair of ethmoid sinuses 27a and 27b, and the nasal septum 42.

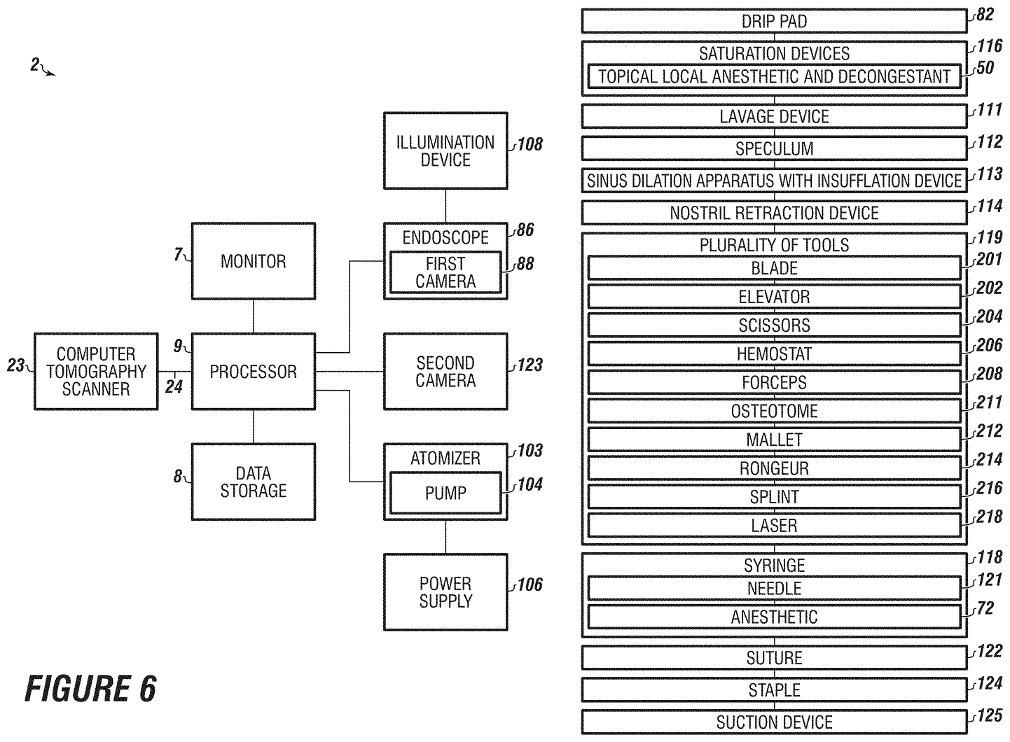

FIG. 6 illustrates a kit with equipment used to implement permanent migraine elimination according to one or more embodiments. In embodiments, the kit 2 can contain equipment for treating the root cause of a migraine via at least one nostril of a patient, which can include a computer tomography scanner 23 (CT scanner) configured to perform the computer tomography scan 24 on at least one sinus cavity of the patient. In embodiments, the equipment can include a processor 9, a data storage 8 and a monitor 7. In embodiments, the kit 2 can contain an atomizer 103 with a pump 104, which can be connected to a power supply 106. For example, the atomizer can be a hand atomizer, a spray bottle, a steam generating device, or a humidifier. In embodiments, the power supply can be 110 AC. In embodiments, the atomizer 103 can be used for spraying a nasal cavity of the patient with a topical local anesthetic and decongestant 50 to form an anesthetized decongested nasal cavity in the patient. An illumination device 108, such as a light emitting diode “LED” with a power supply, can be configured to: (i) enable inspection of the anesthetized decongested nasal cavity to confirm that the proposed medical treatment plan to implement is the best match and (ii) enable inspection between mucosal flaps for a residual broken bone, a residual segment of cartilage, or combinations thereof.

In embodiments, the illumination device can be used to facilitate activity to close the mucosal flaps, sew with suture, staple an incision, dilate the sinus cavities, suction the anesthetized decongested nasal cavity, treat turbinates, or another surgical procedure on the anesthetized decongested nasal cavity. In embodiments, a sinus dilation apparatus with insufflation device 113 can be configured to perform dilation of at least one sinus cavity using the proposed medical treatment plan with the greatest number of matches. In embodiments, the illumination device 108 can be mounted to an endoscope 86. The endoscope 86 can have a first camera 88 for providing images to the data storage 8 connected to the processor 9, which can be in further communication with the monitor 7. In embodiments, a second camera 123 can be configured to take a still image, a video image, or both a still image and a video image prior to therapy or post therapy, and store the image or images in the data storage 8. In embodiments, the equipment can include a drip pad 82, which can be positioned adjacent to at least one nostril of the patient. In embodiments, the equipment can include a plurality of saturation devices 116, such as cottonoids. Each saturation device 116 can be saturated with a topical local anesthetic and decongestant 50.

In embodiments, each saturation device 116 can be used to contact at least one turbinate of the patient to decongest the at least one turbinate. In embodiments, a lavage device 111 can be used for performing a lavage after removing at least partially, at least one ethmoid sinus of the patient or a maxillary sinus of the patient. In embodiments, a speculum 112 and a nostril retraction device 114 can be configured to expose at least one mucosal flap of the nasal septum. In embodiments, the sinus dilation apparatus with insufflation device 113 can be configured to perform inflation on all sinus cavities except ethmoid sinuses of the patient. In embodiments, a syringe 118 with a needle 121 can be used to infuse an anesthetic 72 into the nasal cavity of the patient. For example, from 10 cc to 40 cc of anesthetic can be used on an average adult of 175 kilograms. In embodiments, the equipment can include a plurality of tools 119.

In embodiments, the plurality of tools can include but is not limited to: a blade 201, an elevator 202, a scissors 204, a hemostat 206, a forceps 208, an osteotome 211, a mallet 212, a rongeur 214, a splint 216, and a laser 218. In embodiments, the scissors 204 can be double action scissors or turbinate scissors. In embodiments, the forceps can be bayonet forceps. A portion of the plurality of tools can remove bone and cartilage of the nasal septum, reconstruct cartilage of the nasal septum, reposition at least one of: bone and cartilage of the nasal septum, at least partially remove at least one ethmoid sinus of the patient, and open at least one maxillary sinus of the patient. In embodiments, some of the plurality of tools can be configured to perform at least one operation on the cartilage, the bone or both the cartilage and the bone, comprising: chiseling, cutting, fracturing, incising, and shaving. In embodiments, the laser 218 can be used to burn at least one turbinate. In embodiments, the equipment can include a suture 122 or at least one staple 124 to surgically close the exposed mucosal flaps of the nasal septum. In embodiments, the equipment can include a suction device 125 to remove unwanted matter from the anesthetized decongested nasal cavity.

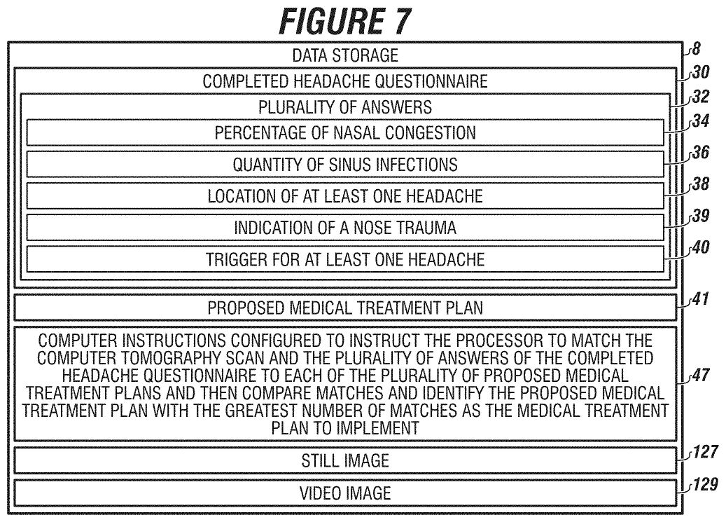

FIG. 7 illustrates a data storage according to one or more embodiments of the disclosure. The term “data storage” refers to a non-transitory computer readable medium, such as a hard disk drive, solid state drive, flash drive, tape drive, and the like. The term “non-transitory computer readable medium” excludes any transitory signals but includes any non-transitory data storage circuitry, e.g., buffers, cache, and queues, within transceivers of transitory signals. In embodiments, the data storage 8 can contain a headache questionnaire, a plurality of answers 32 from the completed headache questionnaire 30, a still image 127 and a video image 129. The plurality of answers 32 can indicate a percentage of nasal congestion 34, such as a range from 0.1 percent to 100 percent, a quantity of sinus infections 36, such as the number experienced by the patient during a preset unit of time, a location of at least one headache 38, an indication of a nose trauma 39, and a trigger for the at least one headache 40. In embodiments, the data storage 8 can contain a plurality of proposed medical treatment plans 41.

In embodiments, the data storage 8 includes computer instructions 47 configured to instruct a processor to match the computer tomography scan and the plurality of answers of the completed headache questionnaire to each of the plurality of proposed medical treatment plans and then compare the matches to identify the proposed medical treatment plan with the greatest number of matches as the medical treatment plan to implement.

Another advantage of the diagnostic process of the present disclosure is that it cures other related or neurological symptoms associated with migraine, such as, but not limited to, seizure, tremor, tingling, paralysis or numbness of arms and legs, facial numbness or weakness, eyelid ptosis, tinnitus, hearing loss, dizziness, aphasia, slurred speech, impaired vision or blindness, light sensitivity, seeing flashing lights, impaired thinking, confusion, nausea and vomiting. These symptoms have been shown to correlate to the nasal/sinus abnormalities or contact conditions described herein and can also be remedied by the diagnostic and subsequent medical procedure described herein.

In another exemplary embodiment of the disclosure a method to use the kit to reduce migraine headaches in a patient is provided. Further, the method when used with the kit and equipment can permanently eliminate headaches, such as migraines in at least 88 percent of patients.? The method for treating a headache, such as a migraine via at least one nostril of a patient can include performing a computer tomography scan (CT Scan) on at least one sinus cavity of the patient. The method can include reviewing a completed headache questionnaire on the patient, the completed headache questionnaire requiring a plurality of answers, the plurality of answers indicating a percentage of nasal congestion from 0.1 percent to 100 percent, a quantity of sinus infections experienced by the patient during a preset unit of time, a location of at least one headache, and a trigger for at least one headache. In embodiments, the plurality of answers can include an indication of a nose trauma. In embodiments, the plurality of answers can be stored in the data storage connected to the processor using an input device connected to the processor, wherein the input device can be a human machine interface, such as a mobile phone application or a computer application. The method can include spraying a nasal cavity of the patient with a topical local anesthetic and decongestant forming an anesthetized decongested nasal cavity. The topical anesthetic and decongestant can be a formulation having 50 weight percent to 80 weight percent of a phenylepinephrine and 20 weight percent to 50 weight percent of at least one of: a lidocaine, oxymethazoline, and a tetracaine. In embodiments, additional components can be added to the formulation and be usable herein. Some of the additional components can be at least one of: 1 weight percent to 5 weight percent of a peppermint oil, 1 weight percent to 5 weight percent of a menthol, 1 weight percent to 10 weight percent of an emu oil, 1 weight percent to 5 weight percent of an eucalyptus oil, 1 weight percent to 5 weight percent of a lemon oil, 1 weight percent to 5 weight percent of a rosemary oil, 1 weight percent to 5 weight percent of a tea tree oil, 1 weight percent to 5 weight percent of a pine oil, 1 weight percent to 5 weight percent of a lavender oil, 1 weight percent to 5 weight percent of a thyme oil, and 1 weight percent to 5 weight percent of a camphor.

### Formulation Example 1

As an example, the formulation can be 60 weight percent of phenylephrine, 30 weight percent of a lidocaine, 3 weight percent of peppermint oil, 4.5 weight percent of emu oil, and 2.5 weight percent of lemon oil.

### Formulation Example 2

As an example, the formulation can be 68 weight percent of phenylephrine, 20 weight percent of a tetracaine, 2 weight percent of menthol, 5 weight percent of tea tree oil, and 4 weight percent of pine oil and 1 weight percent of camphor.

### Formulation Example 3

As an example, the formulation can be 40 weight percent of phenylephrine, 50 weight percent of oxymetazoline, 3.5 weight percent of eucalyptus oil, 1.5 weight percent of rosemary oil, 3 weight percent of lavender oil, and 2 weight percent of thyme oil.

In embodiments, the topical local anesthetic and decongestant can include from 10 weight percent to 20 weight percent of a nasal steroid, such as triamcinalone acetonide.

### Formulation Example 4

As an example, the formulation can be 60 weight percent of phenylephrine, 30 weight percent of a lidocaine and 10 weight percent of triamcinolone acetonide. The method can include inspecting the anesthetized decongested nasal cavity. The method can include developing a proposed medical treatment plan to treat at least one of: a nasal septum, at least one sinus cavity, and at least one turbinate of the patient using information gained from the anesthetized decongested nasal cavity inspection with the computer tomography scan and the plurality of answers to the completed headache questionnaire. The method can include installing topical local anesthetic and decongestant onto at least one turbinate. The method can include infusing an anesthetic into the anesthetized decongested nasal cavity of the patient.

In embodiments, the anesthetic is not a topical anesthetic. For example, the anesthetic can be injectable lidocaine HCL 1 weight percent with epinephrine 1:100,000, such as for infiltration and nerve block. The method can include dilating of at least one nasal cavity using the proposed medical treatment plan and a sinus dilation apparatus with insufflation device. In embodiments, the dilating can be with a balloon or another non-balloon method. In embodiments, sponges or splints can be used to dilate the sinus cavity. The method can include incising at least one of: a first mucosal flap or a second mucosal flap of a nasal septum of the anesthetized decongested nasal cavity to expose cartilage and bone, as shown in box 1800. The method can include removing bone and cartilage of the nasal septum.

The method can involve fracturing at least one turbinate laterally away from the nasal septum. The method can involve inspecting between mucosal flaps for a residual broken bone, a residual segment of cartilage, or combinations thereof. The method can involve surgically closing the exposed mucosal flaps of the nasal septum. The method can involve suctioning unwanted matter from the anesthetized decongested nasal cavity. The method can involve removing at least partially, at least one ethmoid sinus of the patient. The method can involve performing a lavage after at least partially removing at least one ethmoid sinus of the patient.

The method can involve performing a lavage of the nasal cavity after surgically closing the mucosal flaps of the nasal septum. The method can involve performing a lavage after dilating at least one sinus cavity. The method can involve performing at least one of: reconstruction of cartilage of the nasal septum and repositioning of at least one of: bone and cartilage of the nasal septum. The method can involve burning at least one turbinate, or at least partially excising at least one turbinate. In embodiments, any one of the following steps of the method can be performed and in no specific order: The method can include positioning a drip pad adjacent to at least one nostril of the patient. The method can include out-fracturing a medial ethmoid sinus bone while fracturing at least one turbinate laterally away from the nasal septum. The method can include taking a still image, a video image or both a still image and a video image after spraying the nasal cavity, after suctioning, or after any step in between spraying and suctioning.

In embodiments, the dilating can be performed on any number of sinus cavities in a patient, such as at least one sinus cavity or all sinus cavities of the patient. In embodiments, the reconstruction of cartilage can involve performing at least one of: chiseling, cutting, fracturing, incising, and shaving. In embodiments, the inspecting of the anesthetized decongested nasal cavity can be with an illumination device. In embodiments, the illumination device can be mounted to an endoscope. In embodiments, the endoscope can be attached to a camera that connects to a monitor. In embodiments, the unwanted matter can be a member of the group: mucus, blood, pus, irrigation fluid, and combinations thereof. As an example, a patient, Kelvin has debilitating migraine headaches that require him to stay home from work and lay down in a dark room for several hours. To heal Kelvin, the following steps are implemented. First, Kelvin is given an initial consult to determine the extent of his headaches, problems in his head, and the root cause of his migraine headache. In the initial consultation, Kelvin is asked specific headache questions to provide a plurality of answers. Then Kelvin has his nasal cavity sprayed with a sufficient amount of a topical local anesthetic and decongestant, such as a formulation of 50 weight percent of phenylephrine and a 50 weight percent of lidocaine to coat all the nasal cavities. About 0.5 cubic centimeters to 2 cubic centimeters of the topical local anesthetic and decongestant is used for Kelvin.

An illumination device is used with an endoscope having a camera to inspect the anesthetized decongested nasal cavity while projecting the camera images on a monitor. A proposed medical treatment plan is tentatively developed to treat a nasal septum. Kelvin is given a prescription to have a computer tomography scan to confirm the diagnosis developed from the inspection. The resulting computer tomography scan is reviewed, and Kelvin is diagnosed with having chronic sinusitis in addition to a deviated nasal septum with enlarged turbinates. The proposed medical treatment plan is revised in view of the review of the computer tomography scan. A surgical procedure date is planned with the patient. On the day of surgery, a topical local anesthetic and decongestant is installed onto the enlarged turbinates with a waiting period from 2 minutes to 5 minutes.

An anesthetic is injected with syringe and needle into the nasal septum and turbinates. All of Kelvin's sinus cavities except for ethmoid sinuses are dilated with an ACCLARENT™ balloon sinuplasty apparatus, which takes from approximately 8 minutes to 15 minutes. One of the mucosal flaps is incised to expose cartilage and bone and a SMITH™ speculum is used to retract the flap. Offending cartilage and bone, which forms the deviated nasal septum is removed. An inspection is made between the mucosal flaps for broken bone and cartilage segments. Suctioning or removal of bone fragments or cartilage segments is performed. The mucosal flap is surgically closed. Additional suctioning is performed to remove unwanted matter. At least one turbinate is then out-fractured laterally away from the nasal septum. Kelvin experiences a full elimination in migraine headache occurrences.

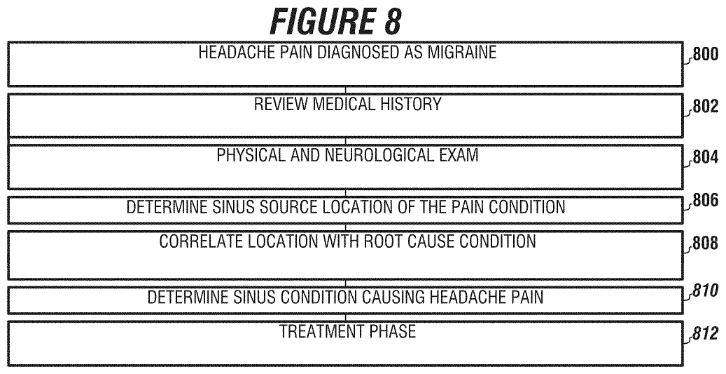

FIG. 8 illustrates an exemplary diagnostic method of the present disclosure. The method begins at step 800 where the headache pain is diagnosed as actual migraine pain. Since there are numerous types of headaches, including tension, cluster, allergy, hormone, caffeine, exertion, and hypertension, for example, it's critical to determine that the headache pain is actually migraine pain and not another type of headache or another medical condition that may present symptoms that are similar to migraine pain. The diagnosing of a migraine may include, for example, a thorough review of a patient's medical history with a focus on prior headaches location, intensity, frequency, and duration, as shown at step 802. At step 804, the patient may undergo a complete physical and neurological exam looking for signs and symptoms of an illness or other condition that causes migraine headaches. Once the headache is determined to be a primary headache (migraine, cluster, or tension headache), then the diagnostic process of the present disclosure continues to step 806, where the method determines the location of the pain condition. Pain location may be determined from patient survey information and/or from physical exam observations. Once the location of the headache pain is determined, then the location of the headache pain is correlated to a source or root cause nasal/sinus location at step 808. Once the location of the headache pain is correlated to a source or root cause nasal/sinus location, then the root cause nasal/sinus location is analyzed to determine a nasal/sinus condition causing the headache pain at step 810. Analyzing the root cause nasal/sinus location analyzing CT scan image slices of the head and general nasal/sinus areas of the patient. These CT scans are analyzed to identify particular physical conditions that the novel method of the present disclosure uses to correlate headache pain and specifically migraine pain to a root cause condition, i.e., specific structural abnormalities in the nose and sinus areas can be correlated via the novel method of the present disclosure to a root cause of specific migraine headache pains.

Analyzing the nose and sinus area on CT scan as part of step 810 is an involved and intricate process that includes determining boundaries of turbinates in the identified nasal locations that are the cause of migraine pain. With the boundaries determined, the method determines if those boundaries touch or otherwise intersect or contact any of the turbinate tissue. Further, diagnostic method of the present disclosure, when analyzing the CT scan images of the various nasal/sinus cavities, is also generally configured to review turbinate shape for abnormalities that have been shown by the inventors to be a root cause of migraine headaches. More particularly, aside from the shape of turbinates causing physical contact with adjacent structures, the shape of turbinates being significantly deformed or altered from the original shape has also been shown by the inventors to be a root cause of migraine headaches. The “elf shoe” turbinate deformity (curvature of the inferior turbinates) is indicative of excessive turbinate tissue that potentially contacts its boundaries and has also been shown by the inventor to be a root cause of migraine headaches. The determination of size and boundaries of turbinates and the surrounding structures at step 810 may be accomplished by a software process or program product.

Once the diagnostic method of the present disclosure identifies the nasal/sinus migraine root cause condition(s), which are generally turbinate contact with adjacent tissue, bone spurs, turbinate deformity, narrow or obstructed ostia of the sinuses with or without sinus disease as described above, then the method of the present disclosure continues to the treatment phase at step 812. The treatment phase generally includes surgically repairing or correcting the identified root cause conditions, including separating turbinates from contacting adjacent structures, trimming deformed or enlarged turbinates to remove the deformity, removing or smoothing bone spurs in the nose and performing balloon sinuplasty on the indicated sinuses. It should be noted, however, that the nasal/sinus root cause conditions causing severe pain may not be considered a migraine by some clinicians, however, despite the terminology used or the selected definition of a migraine, the novel method of the present disclosure allows a medical practitioner to identify nasal/sinus conditions and/or abnormalities that when addressed or corrected with minor surgery can completely eliminate migraine or severe headache pain for the patient.

The types of surgical procedures used to remedy the migraine condition once the root cause is identified may include turbinate trimming or reduction, septoplasty, rhinoseptoplasty, and other nasal/sinus surgical procedures. For a turbinate reduction, the small structures inside the nose that cleanse and humidify air that passes through the nostrils into the lungs are trimmed or otherwise altered to minimize or eliminate contact with adjacent structures in the respective nasal cavity. Turbinates are made up of a bony structure surrounded by sensitive vascular mucous membrane tissue and can become swollen and inflamed by allergies, hormones, irritation or infection, causing nasal obstruction along with producing an excessive amount of mucus which leads to congestion. As these events cause turbinates to change in size, structure, shape, and position, the turbinates contact adjacent structures in the nasal area and become a root cause for a migraine headache. Therefore, turbinate reduction or modification has been shown by the inventor of the present novel method to eliminate migraine headache pain. Radiofrequency turbinate reduction is one available procedure in which a needle-like instrument is inserted into the turbinate and energy is transmitted to the tissue to cause a controlled damage, so by the time healing process occurs, the turbinates will be reduced, allowing improved airflow through the nose and reduced or eliminated contact with surrounding structures in the nasal area. Another type of turbinate reduction or modification surgery is a submucous resection which involves making a small cut in the lining of the turbinates and lifting the lining off the thin bone that forms the structural support of the turbinate. By removing part of this bone and allowing the healing process to shrink the lining of the turbinate, the nasal passage increases in size and turbinate contact with surrounding structures is reduced or eliminated.

Septoplasty is another surgical procedure used to reduce or eliminate identified migraine root cause conditions in the nose. Septoplasty is typically the surgical correction of defects and deformities of the septum, such as deviations or bone spurs. The septum is a structure made of bone and cartilage in the central portion of the nose that separates one nasal cavity from another. When the septum is deviated, it can block one side of the nose and significantly disturb airflow and also make contact with turbinates and adjacent structures, and therefore, be a root cause condition of migraine headache pain. Septoplasty straightens the septum as much as possible in the midline position and opens the airway by removing the deviated portion and maintaining the remaining bone and cartilage in a midline position so as to reduce or eliminate its contact with the turbinates and adjacent structures.

Turbinectomy is another surgical procedure used to reduce or eliminate identified migraine root cause conditions in the nose. In this surgical procedure part of the lower turbinate is trimmed with scissors or reduced with a microdebrider. Similarly, in a turbinoplasty a tool, such as an inflatable bladder or elevator instrument, is placed in the nose to change the position of the turbinate through selective positioning and inflation of the bladder or manual manipulation, which is generally referred to as an outfracture technique to provide space between the turbinate and the septum eliminating potential contact. When a balloon sinus ostial dilation is preformed, under endoscopic visualization, the sinus balloon device is placed into the nasal cavity and guided into the identified root cause sinus area. Once positioning in the root cause sinus area has been confirmed, the balloon is inflated to dilate the sinus opening or drainage tract. Once the respective parts have been moved, dilated or adjusted by inflating the balloon, then the balloon is then deflated and removed and the procedure is complete. There are generally two types of sinus balloon devices, a flexible guidewire assisted device and a malleable rigid device which uses no guidewire.

While these embodiments have been described with emphasis on the embodiments, it should be understood that within the scope of the appended claims, the embodiments might be practiced other than as specifically described herein.

What is claimed is:

1. A diagnostic method for determining if a patient is viable candidate for a surgical procedure to permanently eliminate migraine headache pain, comprising:
determining if a patient's pain is a migraine pain condition or pain from another medical condition;
determining an anatomical location of the determined migraine pain condition;
correlating the determined anatomical location of the migraine pain condition to a root cause nasal/sinus location; and
determining a root cause nasal/sinus condition in the root cause nasal/sinus location that is causing the patient's migraine pain.

2. The diagnostic method of claim 1, wherein determining if a patient's pain is a migraine pain condition or pain from another medical condition comprises an extensive history and physical examination.

3. The diagnostic method of claim 1, wherein determining an anatomical location of the determined migraine pain condition comprises identifying the part of the head or facial area that is a source of the patient's pain.

4. The diagnostic method of claim 2, wherein correlating the determined anatomical location of the migraine pain condition to a root cause nasal/sinus location comprises identifying a specific nasal/sinus area correlating to the anatomical location of the migraine pain condition.

5. The method of claim 4, wherein the specific nasal/sinus area comprises the maxillary sinus, an ethmoid sinus between the eyes, a frontal sinus, a sphenoid sinus, and an area surrounding a septum.

6. The diagnostic method of claim 5, wherein determining a root cause nasal condition in the root cause nasal location that is causing the patient's migraine pain comprises identifying a point of contact between a turbinate in the root cause nasal location that is making contact with adjacent structures.

7. The diagnostic method of claim 5, wherein determining a root cause nasal condition in the root cause nasal location that is causing the patient's migraine pain comprises identifying a turbinate that is oversized or irregularly shaped compared to a normal size or shaped turbinate.

8. The diagnostic method of claim 5, wherein determining a root cause nasal condition in the root cause nasal location that is causing the patient's migraine pain comprises identifying an area of the septum that is either deviated and touching adjacent structures or contains a bone spur that is touching the adjacent structures.

9. The diagnostic method of claim 6, further comprising surgically modifying the turbinate to no longer touch the adjacent structures.

10. The diagnostic method of claim 7, further comprising surgically modifying the irregularly sized turbinate back to a normal size and shape.

11. The diagnostic method of claim 8, further comprising surgically modifying the deviated septum or bone spur to eliminate touching the adjacent structures.

12. The diagnostic method of claim 6, wherein identifying a point of contact between a turbinate in the root cause nasal location that is making contact with an adjacent structures comprises identifying turbinate outer boundaries on a CT scan image, identifying surrounding structures boundaries on the CT scan image, identifying a point where the turbinate outer boundaries intersect with the surrounding structures boundaries.

13. The diagnostic method of claim 12, wherein identifying boundaries on a CT scan image is conducted using pixel differentiation on a computer processing device.

14. The diagnostic method of claim 13, wherein pixel differentiation comprises a computer software package running on a microprocessor that is configured to identify the outer boundaries by comparing pixel color or intensity.

15. A diagnostic method for determining if a patient is viable candidate for a surgical procedure to eliminate migraine headache pain, comprising:
Identifying a head and facial area that is a source of migraine headache pain;
correlating the determined anatomical location of the migraine pain condition to a root cause nasal/sinus location, including identifying a specific nasal/sinus area correlating to the anatomical location of the migraine pain condition; and
determining a root cause nasal/sinus condition in the root cause nasal/sinus location that is causing the patient's migraine pain, including identifying a point of contact between a turbinate in the root cause nasal location that is making contact with adjacent structures.

16. The diagnostic method of claim 15, wherein determining a root cause nasal/sinus condition in the root cause nasal location that is causing the patient's migraine pain comprises identifying a turbinate that is oversized or irregularly shaped compared to a normal size or shaped turbinate.

17. The diagnostic method of claim 15, wherein determining a root cause nasal/sinus condition in the root cause nasal location that is causing the patient's migraine pain comprises identifying an area of the septum that is either deviated and touching adjacent structures or contains a bone spur that is touching the adjacent structures.

18. The diagnostic method of claim 17, further comprising surgically modifying the turbinate to no longer touch the adjacent structures.

19. The diagnostic method of claim 17, further comprising surgically modifying the irregularly sized turbinate to a normal size and shape.

20. The diagnostic method of claim 15, wherein identifying a contact point between a turbinate in the root cause nasal location that is making contact with adjacent structures comprises identifying turbinate outer boundaries on a CT scan image, identifying surrounding structures boundaries on the CT scan image, identifying a point where the turbinate outer boundaries intersect or make contact with the surrounding structures boundaries.
